(19) United States
(12) Patent Application Publication
(10) Pub. No.: US 2026/0182916 A1
(43) Pub. Date: Jul. 2, 2026

(54) WEARABLE DEVICE FOR MEASURING LOCAL IMPEDANCE AND AREA IMPEDANCE USING MULTIPLE ELECTRODES AND CONTROL METHOD THEREOF

(71) Applicant: SAMSUNG ELECTRONICS CO., LTD., Suwon-si (KR)

(72) Inventors: Youngjae OH, Suwon-si (KR); Hyoungseon CHOI, Suwon-si (KR); Taehan JEON, Suwon-si (KR); Seoyoung YOON, Suwon-si (KR); Jinhong MIN, Suwon-si (KR); Jiwoon JUNG, Suwon-si (KR); Taeseon KIM, Suwon-si (KR)

(73) Assignee: SAMSUNG ELECTRONICS CO., LTD., Suwon-si (KR)

(21) Appl. No.: 19/436,663

(22) Filed: Dec. 30, 2025

Related U.S. Application Data
(63) Continuation of application No. PCT/KR2025/023044, filed on Dec. 29, 2025.

(30) Foreign Application Priority Data
Dec. 30, 2024 (KR) 10-2024-0200018

(51) Int. Cl. A61B 5/00; A61B 5/0205; A61B 5/0537; A61B 5/256; A61B 5/282; A61B 5/332; G06F 3/01

(52) U.S. Cl. CPC A61B 5/681; A61B 5/0205; A61B 5/256; A61B 5/282; A61B 5/684; A61B 5/6843; A61B 5/742; G06F 3/011; A61B 5/0537; A61B 5/332

(57) ABSTRACT

There is provided a wearable device including a housing, a display on and partly exposed from the housing, first electrodes on a first surface of the housing, second electrodes on a second surface of the housing, and at least one processor disposed inside the housing. The at least one processor may be configured to detect a contact of the first electrodes to a first position on a user's body, detect a contact of a portion of the user's body to the second electrodes while the plurality of first electrodes contact the first position, measure both a local impedance at the first position and an area impedance including the first position based on the detection of the contact of the first electrodes and second electrodes, and provide information about the user's body through the display based on the measured local impedance and area impedance.

### CROSS-REFERENCE TO RELATED APPLICATIONS

This application is a continuation f International Application No. PCT/KR2025/023044 designating the United States, filed on Dec. 29, 2025, in the Korean Intellectual Property Receiving Office and claiming priority to Korean Patent Application No. 10-2024-0200018, filed on Dec. 30, 2024, the disclosures of each of which are incorporated by reference herein in their entireties.

### BACKGROUND

### Field

The disclosure relates to a wearable device and a control method for measuring local impedance and area impedance using a plurality of electrodes.

### Description of Related Art

More and more services and additional functions are being provided through wearable devices, e.g., smart watches, or other portable electronic devices. To meet the needs of various users and raise use efficiency of wearable devices, communication service carriers or wearable device manufacturers are jumping into competitions to develop wearable devices with differentiated and diversified functionalities. Accordingly, various functions that are provided through wearable devices are evolving more and more.

The above-described information may be provided as related art for the purpose of helping understanding of the disclosure. No claim or determination is made as to whether any of the foregoing is applicable as prior art in relation to the disclosure.

### SUMMARY

According to conventional wearable devices, the impedance measurement function for the user's body area (e.g., upper body) is provided, but the function or operation for measuring impedance for a local area or an area or location desired by the user is not provided.

According to an embodiment of the disclosure, there may be provided a wearable device capable of measuring local impedance as well as area impedance using a plurality of electrodes, thereby measuring the body condition of a local area and implementing more accurate body composition measurement compared to conventional wearable devices.

According to an embodiment of the disclosure, there may be provided a method for controlling a wearable device capable of measuring local impedance as well as area impedance using a plurality of electrodes, thereby measuring the body condition of a local area and implementing more accurate body composition measurement compared to conventional wearable devices.

A wearable device according to an embodiment of the disclosure may comprise a housing, a display disposed on the housing to be at least partially exposed, a plurality of first electrodes disposed on a first surface of the housing, a plurality of second electrodes disposed on a second surface of the housing, and at least one processor disposed inside the housing. The at least one processor may be configured to detect a contact of the plurality of first electrodes to a first position on a user's body, detect a contact of a portion of the user's body to the plurality of second electrodes while the plurality of first electrodes contact the first position, measure both a local impedance at the first position and an area impedance including the first position based on the detection of the contact of the plurality of first electrodes and the plurality of second electrodes, and provide information about the user's body through the display based on the local impedance and the area impedance.

In a computer-readable, non-transitory recording medium configured to store a plurality of instructions, according to an embodiment of the disclosure, the plurality of instructions may include instructions configured to, when executed by at least one processor of a wearable device, enable the wearable device to detect a contact of a plurality of first electrodes of the wearable device to a first position on a user's body, detect a contact of a portion of the user's body to a plurality of second electrodes of the wearable device while the plurality of first electrodes contact the first position, measure both a local impedance at the first position and an area impedance including the first position based on the detection of the contact of the plurality of first electrodes and the plurality of second electrodes, and provide information about the user's body through a display of the wearable device based on the local impedance and the area impedance.

A wearable device according to an embodiment of the disclosure may comprise a housing, a display disposed on the housing to be at least partially exposed, a plurality of first electrodes disposed on a first surface of the housing, a plurality of second electrodes disposed on a second surface of the housing, and at least one processor disposed inside the housing, wherein the at least one processor is configured to measure, using the plurality of first electrodes and the plurality of second electrodes, a local impedance and an area impedance at a plurality of positions, respectively, on a user's body, based on the local impedance and the area impedance, determine whether at least one position among the plurality of positions is a correct position or not; and when the at least one position among the plurality of positions is the correct position, measure the ECG signal at the plurality of positions where the local impedance and the area impedance have been measured.

There is provided a wearable device including a housing; a display disposed on the housing and at least partially exposed from the housing; a plurality of first electrodes disposed on a first surface of the housing; a plurality of second electrodes disposed on a second surface of the housing; and at least one processor disposed inside the housing, and the at least one processor is configured to: detect a first contact of the plurality of first electrodes to a first position on a user's body; detect a second contact of a portion of the user's body to the plurality of second electrodes while the plurality of first electrodes contact the first position; based on detecting the first contact and the second contact, measure both a local impedance at the first position and an area impedance of a combination of the first position and one or more other positions on the user's body; and based on the local impedance and the area impedance, provide, through the display, information about the user's body.

The plurality of first electrodes may include pairs of electrodes.

The at least one processor may be further configured to, based on history information about the local impedance measured at the first position, control the display to provide a guide of a measurement position.

The at least one processor may be further configured to, based on the local impedance, perform a scoring operation on the first position.

The at least one processor may be further configured to control the display to provide a guide of a measurement of the area impedance based on determining that an amount of change in the local impedance of the first position exceeds a designated threshold amount of change.

The at least one processor may be further configured to: determine a body condition at the first position and a second position of the user's body different from the first position and based on the local impedance, the one or more other positions of the user's body comprise the second position; and provide, through the display, an indication of the body condition.

The at least one processor may be further configured to measure the local impedance at the first position using a plurality of frequencies of alternating current (AC) applied to one or more of the plurality of first electrodes.

The at least one processor may be further configured to: determine whether the local impedance measured at the first position exceeds a designated threshold impedance; and based on determining that the local impedance exceeds the designated threshold impedance, output a notification message indicating that a condition of the user's body is changed.

The at least one processor may be further configured to, based on a result of the local impedance and the area impedance, change a baseline of a body composition trend from a first baseline to a second baseline.

There is provided a wearable device including: a housing; a display disposed on the housing and at least partially exposed; a plurality of first electrodes disposed on a first surface of the housing; a plurality of second electrodes disposed on a second surface of the housing; and at least one processor disposed inside the housing, wherein the at least one processor is configured to: measure, using the plurality of first electrodes and the plurality of second electrodes, a local impedance and an area impedance at a plurality of positions, respectively, on a user's body, the local impedance being of a first position on the user's body, the area impedance being of a combination of the first position and one or more other positions on the user's body, and the plurality of positions comprises the first position and the one or more other positions; based on the local impedance and the area impedance, determine whether at least one position among the plurality of positions is a predetermined position; and based on determining that the at least one position among the plurality of positions is the predetermined position, measure an electrocardiogram (ECG) signal at the plurality of positions.

The at least one processor may be further configured to, based on determining that the first position does not correspond to the predetermined position, output a message guiding the wearable device to be moved to another position of the user's body.

There is provided a non-transitory computer-readable recording medium storing a plurality of instructions, wherein the plurality of instructions, when executed by at least one processor of a wearable device, cause the wearable device to: detect a first contact of a plurality of first electrodes of the wearable device to a first position on a user's body; detect a second contact of a portion of the user's body to a plurality of second electrodes of the wearable device while the plurality of first electrodes contact the first position; based on detecting the first contact and the second contact, measure both a local impedance at the first position and an area impedance of a combination of the first position and one or more other positions of the user's body; and based on the local impedance and the area impedance, provide, through a display of the wearable device, information about the user's body.

The plurality of first electrodes may include pairs of electrodes.

The instructions may further cause the wearable device to, based on history information about the local impedance measured at the first position, control the display of the wearable device to provide a guide of a measurement position.

The instructions may further cause the wearable device to, based on the local impedance, perform a scoring operation on the first position.

The instructions may further cause the wearable device to control the display of the wearable device to provide a guide of a measurement of the area impedance based on determining that an amount of change in the local impedance of the first position exceeds a designated threshold amount of change.

The instructions may further cause the wearable device to: determine a body condition at the first position and a second position of the user's body different from the first position and based on the local impedance, the one or more other positions of the user's body comprise the second position; and provide, through the display, an indication of the body condition.

The instructions may further cause the wearable device to measure the local impedance at the first position using a plurality of frequencies of alternating current (AC) applied to one or more the plurality of first electrodes.

The instructions may further cause the wearable device to: determine whether the local impedance measured at the first position exceeds a designated threshold impedance; and based on determining that the local impedance exceeds the designated threshold impedance, output a notification message indicating that a conditions of the user's body is changed.

The instructions may further cause the wearable device to, based on a result of the local impedance and the area impedance, change a baseline of a body composition trend from a first baseline to a second baseline.

### BRIEF DESCRIPTION OF THE DRAWINGS

The above and other aspects, features, and advantages of specific embodiments of the present disclosure will be more apparent from the following description with reference to the accompanying drawings, in which:

FIG. 1 is a block diagram illustrating an electronic device in a network environment according to various embodiments of the disclosure;

FIG. 2 is an example view illustrating a wearable electronic device according to one or more embodiments of the disclosure;

FIG. 3 is an exploded perspective view illustrating an internal structure of a wearable device according to one or more embodiments of the disclosure;

FIG. 4 is an example view illustrating a wearable electronic device according to one or more embodiments of the disclosure;

FIG. 5A is an example view illustrating a lower surface of a wearable device according to one or more embodiments of the disclosure;

FIGS. 5B and 5C are example views illustrating a connection relationship between a plurality of electrodes according to one or more embodiments of the disclosure;

FIGS. 5D, 5E, and 5F are example views illustrating schemes for measuring local impedance and area impedance through a wearable device according to one or more embodiments of the disclosure;

FIG. 5G is a front view illustrating a plurality of electrodes, a biometric sensor module, a wireless charging module, and a magnetic body according to one or more embodiments of the disclosure;

FIG. 6 is an example view illustrating a function or operation of measuring local impedance and area impedance together and providing information about a user's body based on the measured local impedance and area impedance by of a wearable device according to one or more embodiments of the disclosure;

FIG. 7 is an example view illustrating a function or operation of analyzing a state of a user's body portion based on local impedance measured by a wearable device according to one or more embodiments of the disclosure;

FIG. 8 is an example view illustrating a function or operation of determining whether local impedance and area impedance are measured at a correct measurement position, and providing a re-measurement guidance based on the determination result by a wearable device according to one or more embodiments of the disclosure;

FIG. 9 is an example view illustrating a function or operation of performing scoring on a user's body portion based on measured local impedance and area impedance by a wearable device according to one or more embodiments of the disclosure;

FIG. 10 is an example view illustrating the scoring function or operation described in FIG. 9;

FIGS. 11A, 11B, and 11C are example views illustrating a function or operation of determining the degree of a user's muscle development status based on measured local impedance and area impedance by a wearable device according to one or more embodiments of the disclosure;

FIGS. 12A and 12B are example views illustrating a function or operation of measuring local impedance and area impedance at a plurality of positions using a plurality of frequencies, respectively, by a wearable device according to one or more embodiments of the disclosure;

FIG. 13 is a concept view illustrating an electrode structure of a wearable device according to one or more embodiments of the disclosure;

FIG. 14 is an example view illustrating a function or operation of providing information about a possibility of a change in body composition and/or a guide recommending measuring upper body impedance based on local impedance by a wearable device according to one or more embodiments of the disclosure;

FIGS. 15A and 15B are example views illustrating a function or operation of identifying a change in body shape based on measured local impedance and area impedance and providing a guidance message based on the identified change in body shape by a wearable device according to one or more embodiments of the disclosure;

FIG. 16 is an example view illustrating a function or operation of providing feedback on an ECG measurement position by a wearable device according to one or more embodiments of the disclosure;

FIG. 17 is an example view illustrating a table of local impedance and area impedance that a wearable device according to one or more embodiments of the disclosure references to provide feedback on an ECG measurement position;

FIG. 18 is an example view illustrating a scheme for simultaneously measuring leads 1, 2, and 3 for ECG measurement using a wearable device according to one or more embodiments of the disclosure; and

FIG. 19 is an example view illustrating ECG signals exhibiting different waveforms obtained at different positions on a user's body, according to one or more embodiments of the disclosure.

### DETAILED DESCRIPTION

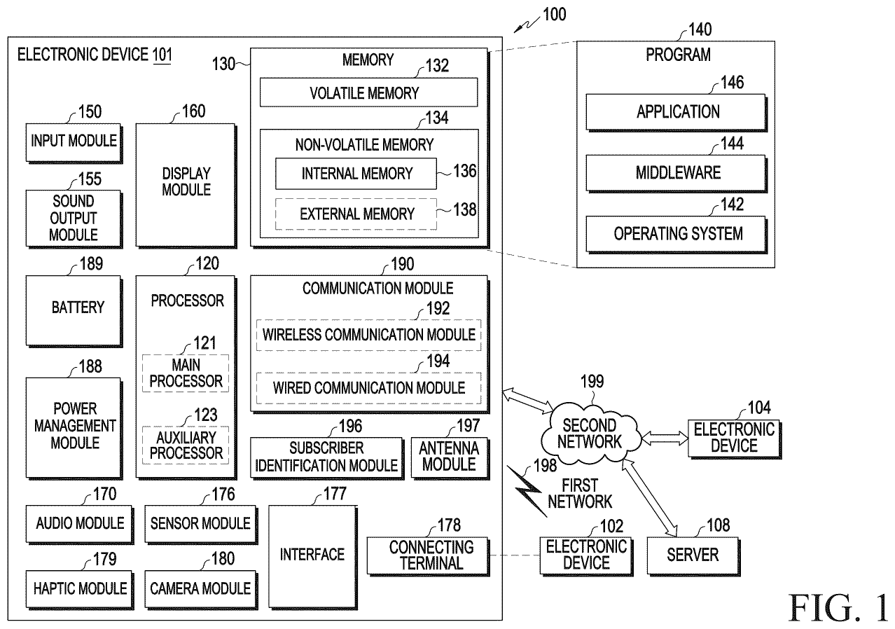

FIG. 1 is a block diagram illustrating an electronic device 101 in a network environment 100 according to various embodiments.

Referring to FIG. 1, the electronic device 101 in the network environment 100 may communicate with at least one of an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In an embodiment, at least one (e.g., the connecting terminal 178) of the components may be omitted from the electronic device 101, or one or more other components may be added in the electronic device 101. According to an embodiment, some (e.g., the sensor module 176, the camera module 180, or the antenna module 197) of the components may be integrated into a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be configured to use lower power than the main processor 121 or to be specified for a designated function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. The artificial intelligence model may be generated via machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by other component (e.g., the processor 120) of the electronic device 101, from the outside (e.g., a user) of the electronic device 101. The input module 150 may include, for example, a microphone, a mouse, a keyboard, keys (e.g., buttons), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display 160 may include, for example, a display, a hologram device, or a projector and control circuitry to control a corresponding one of the display, hologram device, and projector. According to an embodiment, the display 160 may include a touch sensor configured to detect a touch, or a pressure sensor configured to measure the intensity of a force generated by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an accelerometer, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, an HDMI connector, a USB connector, an SD card connector, or an audio connector (e.g., a headphone connector).

The haptic module 179 may convert an electrical signal into a mechanical stimulus (e.g., a vibration or motion) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device 104 via a first network 198 (e.g., a short-range communication network, such as Bluetooth™, wireless-fidelity (Wi-Fi) direct, or infrared data association (IrDA)) or a second network 199 (e.g., a long-range communication network, such as a legacy cellular network, a 5G network, a next-generation communication network, the Internet, or a computer network (e.g., local area network (LAN) or wide area network (WAN)). These various types of communication modules may be implemented as a single component (e.g., a single chip), or may be implemented as multi components (e.g., multi chips) separate from each other. The wireless communication module 192 may identify or authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device). According to an embodiment, the antenna module 197 may include one antenna including a radiator formed of a conductor or conductive pattern formed on a substrate (e.g., a printed circuit board (PCB)). According to an embodiment, the antenna module 197 may include a plurality of antennas (e.g., an antenna array). In this case, at least one antenna appropriate for a communication scheme used in a communication network, such as the first network 198 or the second network 199, may be selected from the plurality of antennas by, e.g., the communication module 190. The signal or the power may then be transmitted or received between the communication module 190 and the external electronic device via the selected at least one antenna. According to an embodiment, other parts (e.g., radio frequency integrated circuit (RFIC)) than the radiator may be further formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mm Wave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. The external electronic devices 102 or 104 each may be a device of the same or a different type from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an Internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or health-care) based on 5G communication technology or IoT-related technology.

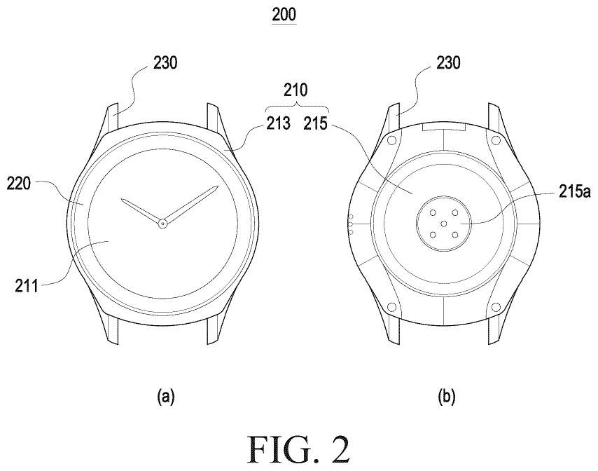

FIG. 2 is an example view illustrating a wearable device (e.g., the electronic device 101) according to one or more embodiments of the disclosure.

The wearable device according to various embodiments of the disclosure may be an electronic devices wearable on the user's body, as well as a portable electronic device, such as a mobile communication terminal. As the electronic device according to various embodiments of the disclosure, a smart watch is described as an example.

Referring to FIG. 2(a) and FIG. 2(b), an electronic device 200 (e.g., a wearable device) according to various embodiments of the disclosure may include a housing 210 including a transparent plate 211, a bezel 220, and a detachment part 230. To describe various embodiments of the disclosure, “first direction” may mean a direction perpendicular to one surface of the transparent plate 211, and “second direction” may mean a direction opposite to the “first direction”.

According to various embodiments, the housing 210 may include a first surface 213 facing the first direction and a second surface 215 facing the second direction opposite the first direction. The front surface of the housing 210 may be opened, and the transparent plate 211 may be mounted to form at least a portion of the first surface 213 corresponding to the front surface of the housing 210 to close the open first surface 213. The first surface 213 and the second surface 215 may have a plate shape and may include a curved surface at an edge portion thereof. The second surface 215 of the housing 210 may include at least one transparent area 215a to emit light generated from an optical element unit (e.g., a light emitting unit) disposed inside the housing to the outside.

According to various embodiments, various circuit devices, such as the processor 120 (e.g., an application processor AP), the memory 130, the input/output interface 150, the communication interface 170, or the like, described above in connection with FIG. 1, may be received in the housing 210, and power may be secured by receiving the battery therein.

According to various embodiments, the housing 210 may be formed of a metal material. According to various embodiments of the disclosure, a portion (e.g., edge) of the housing 210 may be formed of a metal material, and the other portion of the housing 210 may be formed of a plastic material.

According to various embodiments, the transparent plate 211 may be disposed on the first surface 213 of the housing 210. The transparent plate 211 may be formed of a transparent material, e.g., glass or resin (e.g., acrylic or polycarbonate), thereby implementing a screen output from a display device (e.g., 160 of FIG. 1). For example, an analog clock-type screen may be output to the transparent plate 211.

According to various embodiments, the bezel 220 may be disposed at an edge of the transparent plate 211. The bezel 220 may be coupled to the housing 210 to be relatively rotatable so as to rotate along the edge of the transparent plate 211. The bezel 220 may be formed of a metal material to enhance the aesthetic appeal of the electronic device 200. According to one or more embodiments of the disclosure, when the bezel 220 is formed of a metal material, it may be used as an antenna radiator.

According to various embodiments, the detachment part 230 may be disposed to extend and protrude from two opposite ends of the housing 210 in directions away from each other. The detachment part 230 may be coupled to a wearing part (not illustrated) disposed to be worn on the user's wrist. The detachment part 230 may have a binding recess formed to engage with the wearing part. A plurality of binding recesses may be formed in the side surface of the housing 210. The detachment part 230 may have a closed curve shape extending along the circumference of the housing 210. The wearing part may be designed in various forms such as of rubber, plastic, or metal, and these different types of wearing parts may be attached/detached to/from the detachment part 230 of the electronic device 101 based on the user's preference, thereby enhancing the appearance of the electronic device.

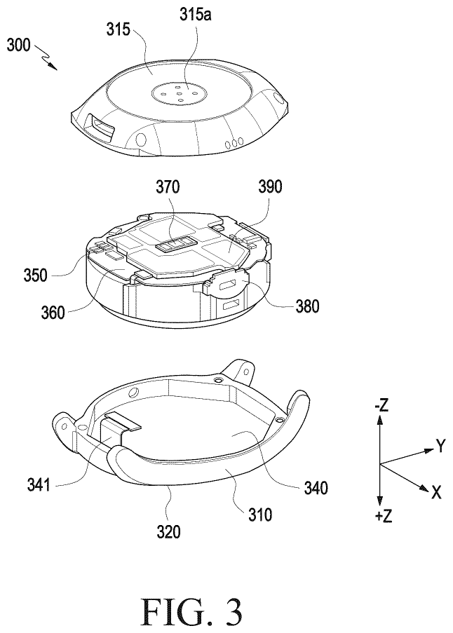

FIG. 3 is an exploded perspective view illustrating an internal structure of a wearable device according to one or more embodiments of the disclosure. In FIG. 3, ‘X’ of the three-axis orthogonal coordinate system may mean a width direction of the electronic device 300, ‘Y’ may mean a length direction of the electronic device 300, and ‘Z’ may mean a thickness direction of the electronic device 300.

Referring to FIG. 3, the electronic device 101 according to one of various embodiments of the disclosure may include a housing 310, a bezel 320, a display 340, an electronic component 350, a main circuit board 360, a bracket 380, the battery, and a biometric sensor 370. The structure of the housing 310 and/or the bezel 320 of the electronic device 300 illustrated in FIG. 3 may be the structure of the housing 210 and/or the bezel 220 illustrated in FIGS. 2A and 2B.

According to various embodiments, the housing 310 may receive various electronic components such as the display 340, the main circuit board 360, the electronic component 350, and/or the biometric sensor 370. A portion of the housing 310, e.g., a side surface of the housing 310, may be at least partially formed of a material that transmits a wireless signal or a magnetic field.

According to various embodiments, the display 340 may be coupled in the second direction (−Z axis direction) of the transparent plate (e.g., 211 of FIG. 2(a). The display 340 may display image information (e.g., photos, videos) to the outside through the transparent plate 211 of FIG. 2(a), and may output execution screens of various applications (e.g., games, Internet banking, schedule management, etc.) according to the user's manipulation.

According to various embodiments, the display 340 may include a liquid crystal display (LCD), a light emitting diode (LED) display, an organic light emitting diode (OLED) display, a microelectromechanical system (MEMS) display, or an electronic paper display. The display 340 may be integrally provided with a touch screen panel to perform a touch screen function. According to various embodiments of the disclosure, the display 340 may perform a wireless communication function by incorporating an antenna radiator on an inner or outer surface thereof.

According to various embodiments, the display 340 may be electrically connected to the display circuit board 341, and the display circuit board 341 may be disposed inside the housing 310. The display circuit board 341 may transmit an electrical signal for display driving 340.

According to various embodiments, the main circuit board 360 may be disposed to face the battery. A processor, a communication module, or the like may be mounted on the main circuit board 360 in the form of an integrated circuit chip. The main circuit board 360 may be electrically connected to the battery. According to various embodiments of the disclosure, the main circuit board 360 may be electrically connected to the electronic component 350 including the antenna radiator or the like through a connector.

According to various embodiments, the electronic component 350 is disposed on the main circuit board 360 and may include an antenna radiator and/or a wireless charging antenna. According to one or more embodiments, the antenna radiator may transmit/receive wireless signals using a magnetic secure transmission (MST) method. For example, the antenna radiator may be an MST antenna. As another example, the antenna radiator may be a near field communication (NFC) antenna that transmits and receives wireless signals through an NFC method. According to one or more embodiments, a shielding structure may be disposed around the antenna radiator to block signal interference between other electronic components such as a sensor module.

According to various embodiments, the wireless charging antenna may be attached to one surface of the main circuit board 360. The wireless charging antenna may be formed in the form of a flat coil. The wireless charging antenna may be formed of a conductive material and may be electrically connected to the main circuit board 360. The wireless charging antenna may generate a current by electromagnetic induction generated from an external electronic device. The current generated by the wireless charging antenna may charge the battery through the main circuit board 360.

According to various embodiments, a heat dissipation structure may be provided between the main circuit board 360 and the battery. For example, the heat dissipation structure may receive heat generated from the main circuit board 360 to prevent the main circuit board 360 from being overheated.

According to various embodiments, a shielding structure 390 may be disposed between the main circuit board 360 and the second surface 315. The shielding structure 390 may shield a space between the electronic component on the main circuit board 360 and the biometric sensor 370 to prevent mutual interference.

According to various embodiments, the second surface 315 formed in the second direction (−Z-axis direction) of the housing 310 may form a rear cover of the housing 310. The rear cover may be formed of a glass material. The rear cover may contact a body portion (e.g., wrist). According to various embodiments of the disclosure, the rear cover is not limited to a glass material, and may be formed of a transparent material such as transparent reinforced plastic. The rear cover may be formed of a transparent plate in a central area to perform the sensing operation of the biometric sensor 370, and formed of an opaque plate in the remaining area. The rear cover may include at least one transparent area 315a for emitting light generated from the internal optical element unit to the outside.

According to various embodiments, the biometric sensor 370 may be disposed between the main circuit board 360 and the second surface 315 to sense biometric information about the user. For example, the biometric sensor 370 may include a heart rate monitoring (HRM). The bio-sensor 370 may detect vasoconstriction/vasodilation based on the reflection of light corresponding to the change in the blood volume within the vessel in the skin of the body. The processor (e.g., the processor 120 of FIG. 1) may receive an electrical signal from the biometric sensor 370 and calculate a heartbeat.

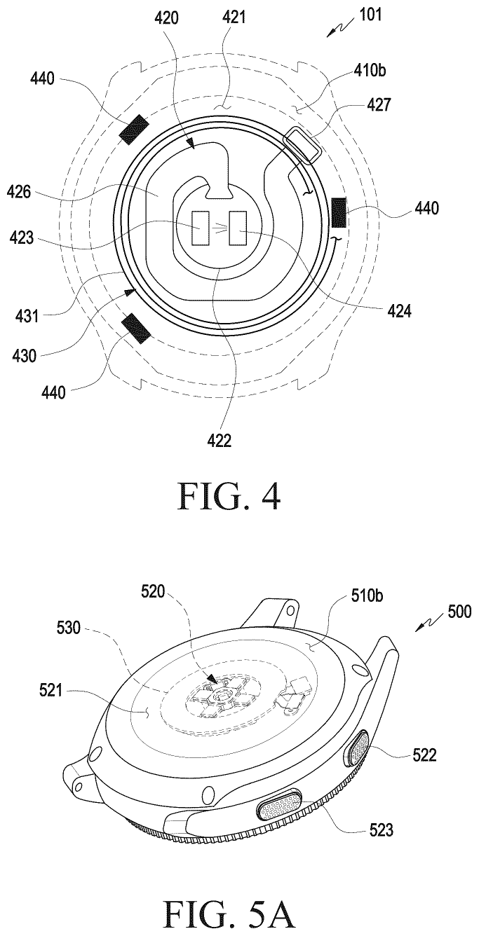

FIG. 4 is an example view illustrating a wearable device (e.g., the electronic device 101) according to one or more embodiments of the disclosure.

Referring to FIG. 4, the biometric sensor module 420 according to one or more embodiments of the disclosure may include a substrate 422, and a light emitting unit 423 and a light receiving unit 424 on the substrate 422. A circuit structure 426 may be connected to one side of the substrate 422, and the circuit structure 426 may extend toward the edge of the second surface 421 to be connected to other electronic components (e.g., power supply and/or processor) through the connector 427.

The battery charging module 430 according to one or more embodiments of the disclosure may have the form of a flat coil, and may generate a current by electromagnetic induction generated from an external electronic device (e.g., a wireless charging pad). The electronic device 101 may charge the battery (not illustrated) embedded in the electronic device 101 using a current generated by the wireless charging antenna. At least one magnetic body 440 may be disposed around the battery charging module 430. The mounting stability of electronic device 101 may be enhanced through the use of at least one magnetic body 440, ensuring that the battery charging operation is conducted stably during charging.

According to one or more embodiments, as illustrated in FIG. 4, the biometric sensor module 420, the battery charging module 430, and the magnetic body 440 may be sequentially disposed from the central portion on the second surface 421 of the rear plate 410b. Three magnetic bodies 440 may be provided, which may be radially disposed to increase mounting stability, e.g., as illustrated in FIG. 4.

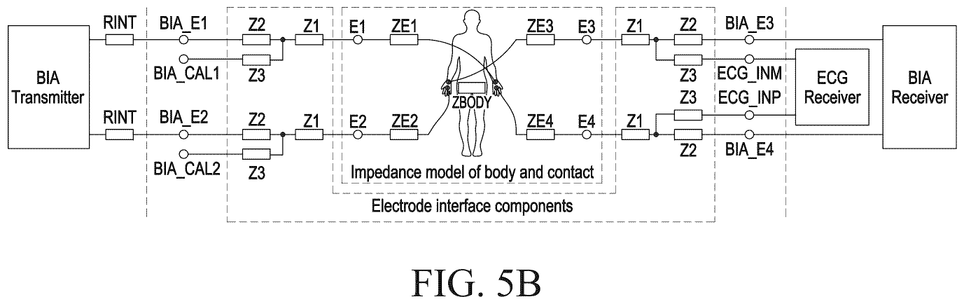

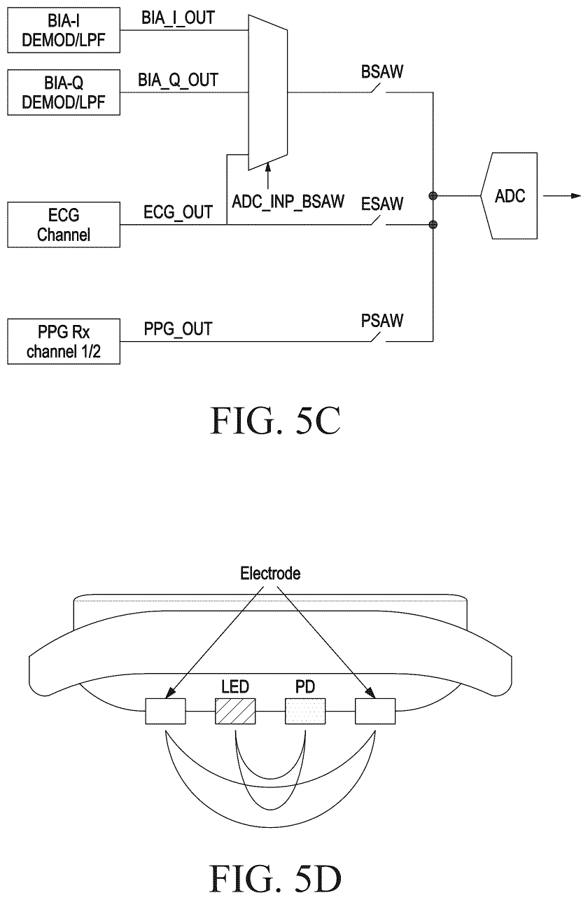

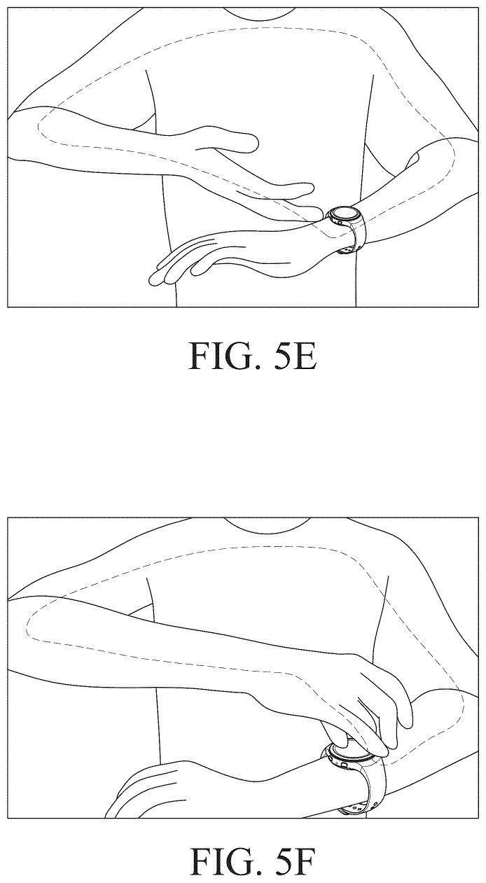

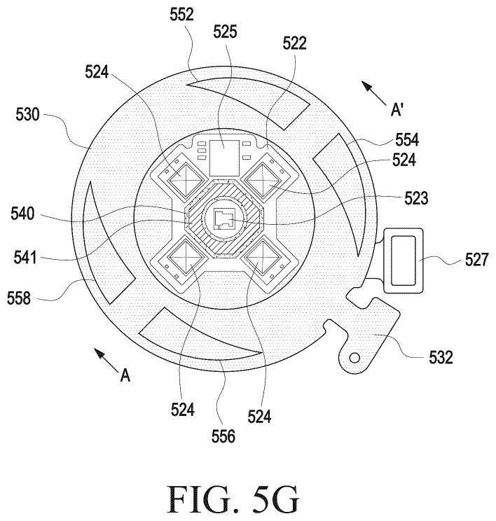

FIG. 5A is an example view illustrating a lower surface of a wearable device according to one or more embodiments of the disclosure. FIGS. 5B and 5C are example views illustrating a connection relationship between a plurality of electrodes according to one or more embodiments of the disclosure.

Referring to FIG. 5A, the electronic device 101 according to various embodiments of the disclosure may include a biometric sensor module 520 and a wireless charging module 530 disposed adjacent to the second surface 521 of the rear plate 510b. The biometric sensor module 520 may be disposed in a central portion of the second surface 521, and the wireless charging module 530 may be disposed to surround the biometric sensor module 520. According to one or more embodiments, at least a portion of the biometric sensor module 520 and at least a portion of the wireless charging module 530 (e.g., a flat coil mounting portion) may overlap each other.

According to various embodiments, the biometric sensor module 520 may be disposed in a space formed by the front plate and the rear plate 510b, e.g., between the main circuit board and the second surface 521, to sense biometric information about the user.

The biometric sensor module 520 may be, e.g., a sensor that collects or measures one or more biometric signals from the user. The biometric sensor module 520 may collect basic data (raw data) for measuring one or more of the user's blood pressure, blood flow, heart rate (HRM, (heart rate variability) HRV), body temperature, respiratory rate, oxygen saturation (SpO2), cardiopulmonary sound detection, blood sugar, waist circumference, height, weight, body fat, calorie consumption, brain waves, voice, skin resistance, electromyogram, electrocardiogram, gait, ultrasound image, sleep state, facial expression (face), pupil dilation, or blinking.

According to one or more embodiments, the electronic device 101 may generate biometric information (or biometric characteristic information) by analyzing the biometric signal. For example, a pulse wave signal obtained through a heart rate variability HRV or a HRM sensor may be the biometric signal. The electronic device 101 may obtain primary biometric information such as average heart rate or heart rate distribution by analyzing the biometric signal, or processing such biometric information to obtain secondary biometric information such as a higher-order stress state or vascular aging.

According to one or more embodiments, the biometric sensor module 520 may simply output the collected user biometric signals, and the biometric sensor module 520 may output biometric information by analyzing the biometric signals through a built-in processor. Therefore, the biometric signal collected through the biosensor module 520 may be transmitted to the processor in the biosensor module 520, a processor (e.g., 120 of FIG. 1) of the electronic device 101 where the biosensor module is embedded, or a processor of an external device (e.g., the server 106 or the electronic device 104 of FIG. 1) to be used to produce biometric information.

When the electronic device 101 where the biometric sensor module 520 is embedded transmits the biometric signal to the remote device (e.g., the electronic device 104 of FIG. 1) or a server (e.g., the server 106 of FIG. 1) through a wired network, a wireless network, or a direct connection, the remote device or server that has received the biometric signal may process the biometric signal to generate biometric information. According to one or more embodiments, when the electronic device 500 where the biometric sensor module 520 is embedded generates primary biometric information and transmits the generated biometric information to the remote device or server, secondary biometric information may be generated from the remote device or server.

For example, the biometric signals collected by the HRM sensor or HRV sensor embedded in the electronic device 101 are transmitted to a smartphone (e.g., a host or main electronic device) wirelessly connected to the electronic device 101, and the smartphone may generate biometric information by analyzing the received biometric signals. The biometric information may be displayed on the display of the smartphone or may be transmitted using a wired or wireless communication means to be displayed on the display of the wrist watch device. The biometric information may be displayed or stored in one or more of, e.g., the smartphone or wrist watch device. According to one or more embodiments, biometric signals gathered by the HRV sensor embedded in the ear clip with earphone functionality may be transferred to the wrist watch device or smartphone, and the electronic device 101 or smartphone may generate biometric information. The generated biometric information may be transferred to one or more devices. If the smartphone generates the biometric information, the electronic device receiving the biometric information may display the information, and the ear clip receiving the biometric information may provide the same to the user through a voice.

According to one or more embodiments, the biometric sensor module 520 may include a heart rate sensor. For example, as the heart repeatedly contracts or relaxes, the peripheral blood vessel varies in blood flow and volume. The photoplethysmography (PPG), one of heart rate sensors, is a technique showing in a waveform the heart beat by measuring the amount of transmitted light using an optical sensor and may measure a variation in the amount of blood in a blood vessel or to SpO2. A heart rate sensor is embedded in, e.g., a clip, wrist watch, necklace, band, or portable phone and may measure biometric signals by attaching or contacting a body portion (e.g., an ear, wrist, carotid, finger, or ankle). As an example, when measurement is performed through a finger, the finger is brought in contact with the heart rate sensor consisting of a light emitter and a light receiver and remains contacting for a predetermined time or longer. Then, the heart rate sensor measures the biometric signal using such a variation that more blood is gathered in the finger during contraction so that the amount of light transmitted through the finger reduces while the blood escapes from the finger during relaxation so that the amount of light transmitted through the finger increases.

The biometric sensor module 520 (e.g., a heart rate sensor) may detect the amount of light penetrating the finger as a voltage. Further, the biometric sensor module 520 (e.g., a heart rate sensor) or the electronic device 101 may convert the detected voltage into a digital value and measure the frequency at which the change occurs. The biometric sensor module 520 (e.g., a heart rate sensor) or the electronic device 101 may be aware of the number of pulses generated per second based on the detected voltage and may compute the heart rate or elapsing time between heart beats using the same. When a PPG sensor, as the heart rate sensor, is embedded in the electronic device (e.g., electronic device 101), a biometric signal may be detected through the radial artery or ulnar artery, and even not with the arteries, a biometric signal may be measured through where vessels are distributed. Further, since a signal generated from the heart has a delay in being transferred to each portion of the body, a difference may occur between the ECG signal and the heart rate signal. For example, when the heart rate sensor is mounted in the wrist watch device or ear clip, a time delay may arise when the signal delivers from the heart to a wrist or ear. The per-minute heart rate varies depending on the examiner's age, and the heart rate pattern may differ depending on emotional states. The electronic device 101 may measure the vessel elasticity through pulse wave analysis and may determine the aging degree of vessel through the same. That is, the electronic device may analyze the strength of cardiac output, vessel elasticity, or amount of remaining blood through accelerated plethysmograph (APG) analysis obtained by performing quadratic differential on the pulse wave signal and may perform an auxiliary test on, e.g., high blood pressure, diabetes, high blood fat, arteriosclerosis, heart disease, or peripheral blood circulatory disturbance by automatically analyzing, e.g., blood vessel elasticity or hardening degree through the same. The wearable device according to one or more embodiments of the disclosure may include an electrode provided on at least one button (e.g., a first button 522 and/or a second button 523) provided on a side surface. The wearable device according to one or more embodiments of the disclosure may measure the area impedance based on the user's contact with the electrode provided on the at least one button (e.g., the first button 522 and/or the second button 523) and the user's contact with at least some of the plurality of electrodes (e.g., the first electrode 552, the second electrode 554, the third electrode 556, and/or fourth electrode 558). As illustrated in FIGS. 5B and 5C, the wearable device 200 according to one or more embodiments of the disclosure may include a plurality of electrodes (e.g., first electrode ZE1, 552, second electrode ZE2, 554, third electrode ZE3, 556, and/or fourth electrode ZE4, 558) on the rear surface of the wearable device 200. The wearable device 200 according to one or more embodiments of the disclosure may identify that contact of the user's skin with at least four electrodes is required, as illustrated in FIGS. 5B and/or 5C, when it is configured to obtain biometric information using bioelectrical impedance analysis (BIA) (e.g., when it is configured to measure the local impedance and/or area impedance). The wearable device 200 according to one or more embodiments of the disclosure may identify that in order to measure the ECG, contact of the user's skin with at least two electrodes is required. Accordingly, the wearable device 200 according to one or more embodiments of the disclosure may indicates to the user that it is required to contact the side electrodes (e.g., the electrodes provided on the at least one button (e.g., the first button 522 and/or the second button 523)) to obtain biometric information through the BIA, while the plurality of electrodes (e.g., the first electrode ZE1, 552, the second electrode ZE2, 554, the third electrode ZE3, 556 and/or the fourth electrode ZE4, 558) disposed on the rear surface of the wearable device 200 are in contact with the skin. Alternatively, the wearable device 200 according to one or more embodiments of the disclosure may indicate to the user that the user is required to contact at least one of the side electrodes (e.g., the electrodes provided on the at least one button (e.g., the first button 522 and/or the second button 523)) in order to measure the ECG. The wearable device 200 according to one or more embodiments of the disclosure may control at least one switching device (e.g., bulk surface acoustic waves (SAW) (BSAW), electrochemical-SAW (ESAW), and/or pseudo-SAW (PSAW)) to individually or simultaneously perform a function of obtaining biometric information or measuring ECG through BIA. For example, the wearable device 200 according to one or more embodiments of the disclosure may continuously perform BIA-based biometric information measurement and ECG measurement based on the same measurement posture when detecting the user's contact with all of the side electrodes (e.g., the electrodes provided on the at least one button (e.g., the first button 522 and/or the second button 523)) while the rear electrodes (e.g., the first electrode ZE1, 552, the second electrode ZE2, 554, the third electrode ZE3, 556 and/or the fourth electrode ZE4, 558) are in contact with the skin. In other words, the electrode for measuring biometric information through BIA and the electrode for measuring ECG may be the same electrode.

FIGS. 5D, 5E, and 5F are example views illustrating methods for measuring local impedance and area impedance through a wearable device 200 according to one or more embodiments of the disclosure.

Referring to FIGS. 5D, 5E, and 5F, the wearable device 200 according to one or more embodiments of the disclosure may be configured to measure the local impedance using at least four electrodes (e.g., the first electrode 552, the second electrode 554, the third electrode 556, and/or the fourth electrode 558). Alternatively, the wearable device 200 according to one or more embodiments of the disclosure may be configured to measure the area impedance using at least four electrodes (e.g., at least two rear electrodes and at least two side electrodes). When measuring the local impedance, the wearable device 200 according to one or more embodiments of the disclosure may be configured to apply current (e.g., several mA) to one first pair of electrodes (e.g., the first electrode 552 and the second electrode 554) among the rear electrodes (e.g., the first electrode 552, the second electrode 554, the third electrode 556 and/or the fourth electrode 558) and, for the other remaining second pair of electrodes (e.g., the third electrode 556 and the fourth electrode 558), measure the difference between voltages applied to the other remaining pair of electrodes according to the application of the current. The wearable device 200 according to one or more embodiments of the disclosure may measure the local impedance based on the intensity of the applied current and the voltage difference. For example, the wearable device 200 according to one or more embodiments of the disclosure may measure local impedance through a designated formula configured to calculate impedance according to the applied current and voltage difference, or through a lookup table that defines the relationship between current intensity, voltage difference, and local impedance value. The wearable device 200 according to one or more embodiments of the disclosure may be configured to change the electrodes constituting the pair of electrodes using at least one switching element provided in the wearable device 200. For example, the wearable device 200 according to one or more embodiments of the disclosure may be configured to change the electrodes belonging to the first pair of electrodes to the first electrode 552 and the third electrode 556 through the control of the switching element.

FIG. 5G is a front view illustrating a plurality of electrodes, a biometric sensor module, a wireless charging module, and a magnetic body according to one or more embodiments of the disclosure.

According to the embodiment illustrated in FIG. 5G, one light emitting unit 523 is disposed in the central area of the substrate 522, and four light receiving units 524 are radially disposed on the same substrate 522 on which the light emitting unit 523 is disposed at a predetermined distance from the light emitting unit 523. Although FIG. 5B illustrates that four light receiving units 524 are provided, the number of light receiving units 524 and the angle between the plurality of light receiving units 524 may vary according to various embodiments. The electronic device 101 (e.g., the wearable device) according to one or more embodiments of the disclosure may include a plurality of electrodes (e.g., the first electrode 552, the second electrode 554, the third electrode 556, and/or the fourth electrode 558). At least some (e.g., the first electrode 552 and the fourth electrode 558) of the plurality of electrodes according to one or more embodiments of the disclosure may be electrically connected to each other. The electronic device 101 (e.g., a wearable device) according to one or more embodiments of the disclosure may be configured to measure impedance (e.g., local impedance) at the position where at least some (e.g., the first electrode 552 and the fourth electrode 558) of the plurality of electrodes come into contact with the user's skin.

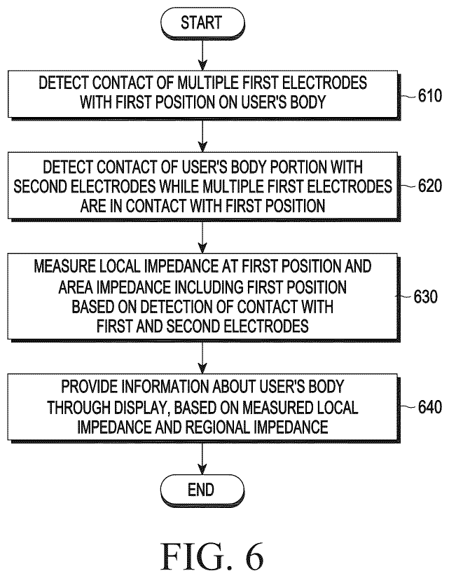

FIG. 6 is an example view illustrating a function or operation of measuring local impedance and area impedance together and providing information about a user's body based on the measured local impedance and area impedance by of a wearable device (e.g., the electronic device 101) according to one or more embodiments of the disclosure.

Referring to FIG. 6, in operation 610, the electronic device 101 (e.g., a wearable device) according to one or more embodiments of the disclosure may detect that the plurality of first electrodes (e.g., the first electrode 552, the second electrode 554, the third electrode 556, and/or the fourth electrode 558) come into contact with a first position (e.g., a wrist) of the user's body. In operation 620, the electronic device 101 (e.g., a wearable device) according to one or more embodiments of the disclosure may detect that a portion (e.g., the user's finger) of the user's body contacts the second electrodes (e.g., the electrode provided on at least one button (e.g., the first button 522 and/or the second button 523)) while the plurality of first electrodes contact the first position. According to one or more embodiments of the disclosure, the electrodes may be disposed in areas (e.g., outer housing and/or bezel areas) other than the area where the button (e.g., the first button 522 and/or the second button 523) is provided. In this case, the wearable device 200 according to one or more embodiments of the disclosure may be configured to measure local impedance and/or area impedance based on the user's contact with the bezel area while at least one of the rear electrodes (e.g., the first electrode 552, the second electrode 554, the third electrode 556, and/or the fourth electrode 558) contacts the user's skin.

The electronic device 101 (e.g., the wearable device 200) according to one or more embodiments of the disclosure may measure the local impedance at the first position and the area impedance including the first position together based on the detection of the contact with the first electrodes and the second electrodes in operation 630. The first position according to one or more embodiments of the disclosure may be input through the electronic device 101 by the user, or the electronic device 101 may estimate the first position based on the impedance history. For example, the electronic device 101 according to one or more embodiments of the disclosure may estimate (or determine) the first position by comparing the previously measured local impedance and/or area impedance with the current impedance measured by the plurality of electrodes.

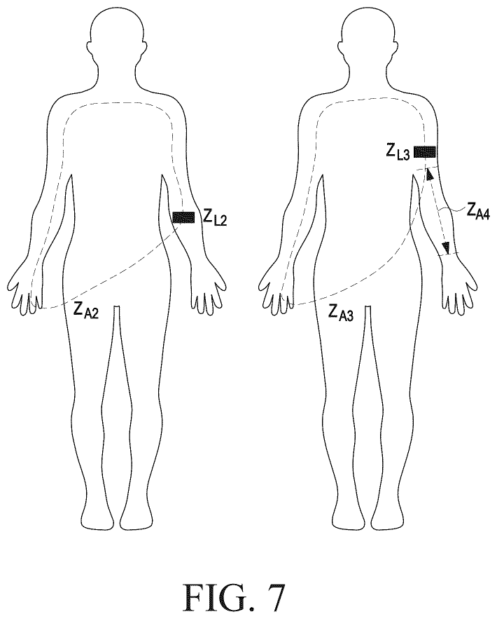

FIG. 7 is an example view illustrating a function or operation of analyzing a state of a user's body portion based on local impedance measured by a wearable device (e.g., the electronic device 101) according to one or more embodiments of the disclosure.

Referring to FIG. 7, the wearable device (e.g., the electronic device 101) according to one or more embodiments of the disclosure may estimate the body composition and muscle mass between the first position and the second position based on the difference between the area impedance value measured at the first position and the area impedance value measured at the second position. The wearable device (e.g., the electronic device 101) according to one or more embodiments of the disclosure may provide information about the estimated body composition and muscle mass between the first position and the second position through the electronic device 101.

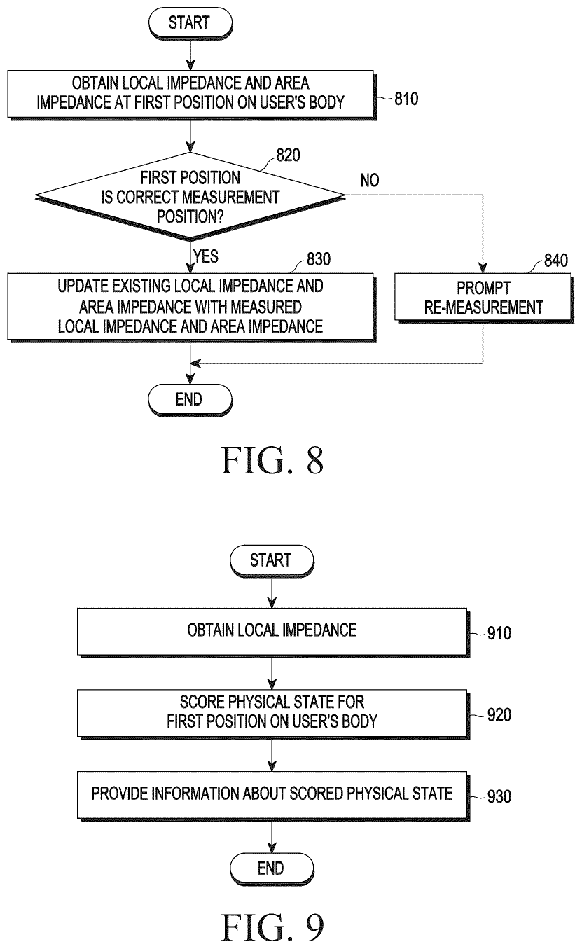

FIG. 8 is an example view illustrating a function or operation of determining whether local impedance and area impedance are measured at a correct measurement position, and providing a re-measurement guidance based on the determination result by a wearable device (e.g., the electronic device 101) according to one or more embodiments of the disclosure.

Referring to FIG. 8, in operation 810, the electronic device 101 according to one or more embodiments of the disclosure may obtain the local impedance and the area impedance at the first position of the user's body. In operation 820, the electronic device 101 according to one or more embodiments of the disclosure may determine whether the first position is a correct measurement position, a predetermined position. The first position according to one or more embodiments of the disclosure may be input through the electronic device 101 by the user, or the electronic device 101 may estimate the first position based on the impedance history. For example, the electronic device 101 according to one or more embodiments of the disclosure may estimate (or determine) the first position by comparing the previously measured (e.g., initially measured) local impedance and/or area impedance with the current impedance measured by the plurality of electrodes. When it is determined that the user wants to measure the local impedance and/or the area impedance at the first position (e.g., the wrist portion) of the user's body (e.g., when the user designates the first position through the electronic device 101), the electronic device 101 may indicate the correct position corresponding to the first position. For example, the electronic device 101 according to one or more embodiments of the disclosure may indicate the correct position by displaying the designated position through the display of the electronic device 101. When the electronic device 101 according to one or more embodiments of the disclosure determines that the electronic device 101 is positioned at the indicated position (e.g., including the surroundings of the position), the electronic device 101 may measure the local impedance and/or the area impedance on the first position where the electronic device 101 is placed. The electronic device 101 according to one or more embodiments of the disclosure may determine whether the electronic device 101 is placed at the correct position using an impedance value (e.g., the local impedance value and/or the area impedance value) corresponding to information about the standard body shape (e.g., standard weight and/or height) determined based on the user's body information. For example, the electronic device 101 according to one or more embodiments of the disclosure may determine whether the electronic device 101 is placed at the correct position by comparing the area impedance value stored in the electronic device 101, which was measured near the wrist, with area impedance value newly measured near the wrist (e.g., the “first position”). The electronic device 101 according to one or more embodiments of the disclosure may store the impedance value newly measured at the first position as a “reference value”.

In operation 840, when the position (e.g., wrist) input by the user and the first position do not match (operation 820-No), the electronic device 101 according to one or more embodiments of the disclosure may provide a re-measurement guidance message. For example, the electronic device 101 according to one or more embodiments of the disclosure may output a guidance message for prompting to reposition the electronic device 101 and measure impedance through the display.

The electronic device 101 according to one or more embodiments of the disclosure may update the existing local impedance and area with the measured local impedance and area impedance when determining that the first position is the correct position in operation 830 (operation 820-Yes). Further, the electronic device 101 according to one or more embodiments of the disclosure may provide designated feedback to the user using the updated impedance value. For example, the electronic device 101 according to one or more embodiments of the disclosure may notify that fat has increased among the body components constituting the upper body when the area impedance value at the first position is increased, and may provide a guidance message for suggesting weight loss through the display. Alternatively, e.g., the electronic device 101 according to one or more embodiments of the disclosure may provide history information about a change in the body shape of the upper body for a designated period through the display, based on the area impedance value.

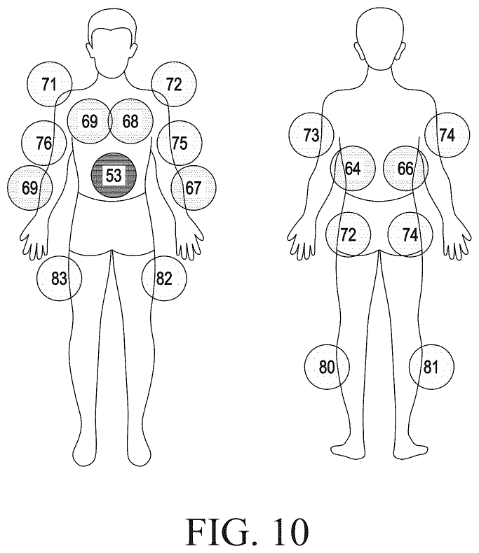

FIG. 9 is an example view illustrating a function or operation of performing scoring on a user's body portion based on measured local impedance and area impedance by a wearable device (e.g., the electronic device 101) according to one or more embodiments of the disclosure. FIG. 10 is an example view illustrating the scoring function or operation described in FIG. 9.

Referring to FIGS. 9 and 10, the electronic device 101 according to one or more embodiments of the disclosure may obtain local impedance in operation 910. The electronic device 101 according to one or more embodiments of the disclosure may be configured to measure the local impedance using at least four electrodes (e.g., the first electrode 552, the second electrode 554, the third electrode 556, and/or fourth electrode 558). When measuring the local impedance, the electronic device 101 according to one or more embodiments of the disclosure may be configured to apply current (e.g., several mA) to one first pair of electrodes (e.g., the first electrode 552 and the second electrode 554) among the rear electrodes (e.g., the first electrode 552, the second electrode 554, the third electrode 556 and/or the fourth electrode 558) and, for the other remaining second pair of electrodes (e.g., the third electrode 556 and the fourth electrode 558), measure the difference between voltages applied to the other remaining pair of electrodes according to the application of the current. The wearable device 200 according to one or more embodiments of the disclosure may measure the local impedance based on the intensity of the applied current and the voltage difference.

In operation 920, the electronic device 101 according to one or more embodiments of the disclosure may score a physical state for the first position of the user's body. For example, the electronic device 101 according to one or more embodiments of the disclosure may estimate muscle mass based on the measured local impedance and perform a scoring operation on the first position, as illustrated in FIG. 10, using a lookup table that defines the relationship between the estimated muscle mass and the score. The electronic device 101 according to one or more embodiments of the disclosure may perform a scoring operation using an impedance value (e.g., the local impedance value and/or the area impedance value) corresponding to information about the standard body shape determined based on the user's body information. The electronic device 101 according to one or more embodiments of the disclosure may compare the impedance value corresponding to information about the standard body shape with the impedance value measured by the electronic device 101. The electronic device 101 according to one or more embodiments of the disclosure may perform the scoring operation using a lookup table where the relationship between the ratio of the impedance value corresponding to information about the standard body shape and the impedance value measured by the electronic device 101, and the score is defined. For example, when the calculated ratio is 0.8, (e.g., when the measured impedance value is smaller than the standard impedance value), the electronic device 101 according to one or more embodiments of the disclosure may determine that the muscle mass for the first position is larger than that of other people, and assign a relatively high score to the first position. According to one or more embodiments of the disclosure, as muscle mass increases, the impedance value (e.g., the local impedance value) may tend to be measured as relatively low. Therefore, the electronic device 101 according to one or more embodiments of the disclosure may determine that the muscle mass for the first position is larger than that of other people when the calculated ratio is 0.8 (e.g., when the measured impedance value is smaller than the standard impedance value).

The electronic device 101 according to one or more embodiments of the disclosure may provide information about the physical state where scoring has been performed in operation 930. When the measured area impedance (e.g., area impedance for the upper body) is higher than the average area impedance for the same/similar age group, the electronic device 101 according to one or more embodiments of the disclosure may estimate that the upper body exhibits a body shape with a higher fat content. Further, the electronic device 101 according to one or more embodiments of the disclosure may estimate that the body type has a high fat content in the abdomen and relatively lean arms when the local impedance near the wrist is lower than the average area impedance for the same or similar age group. In this case, the electronic device 101 according to one or more embodiments of the disclosure may provide a user-customized exercise guide such as an arm strengthening exercise proposal.

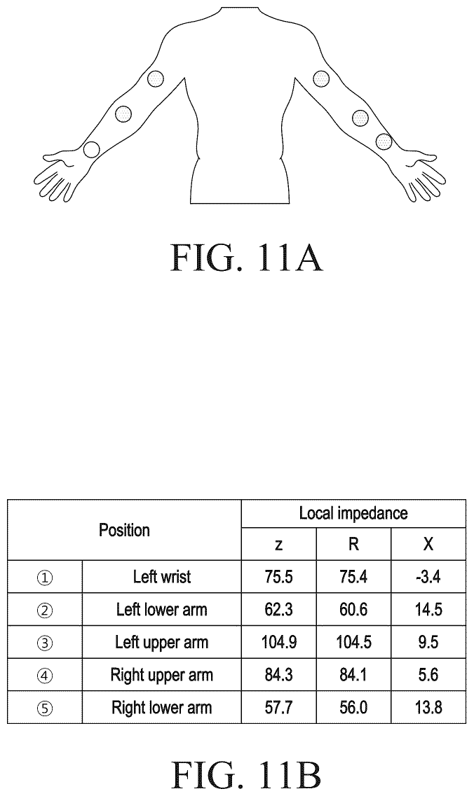

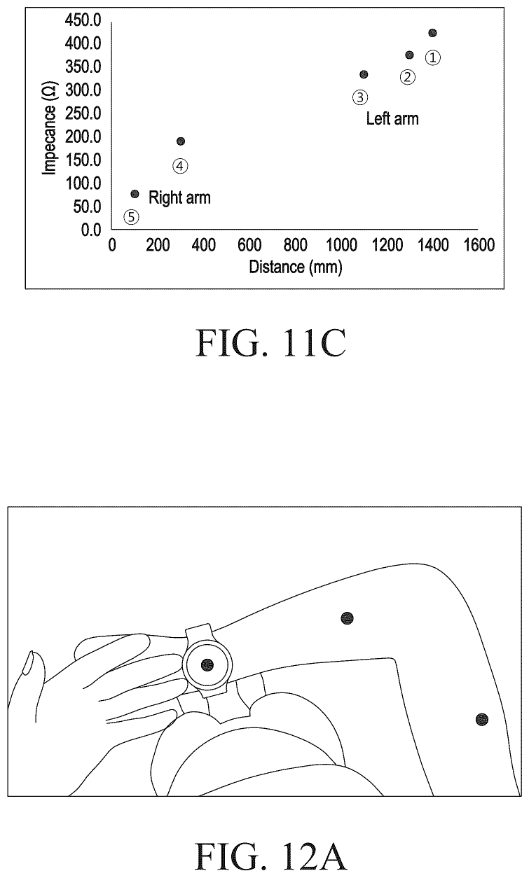

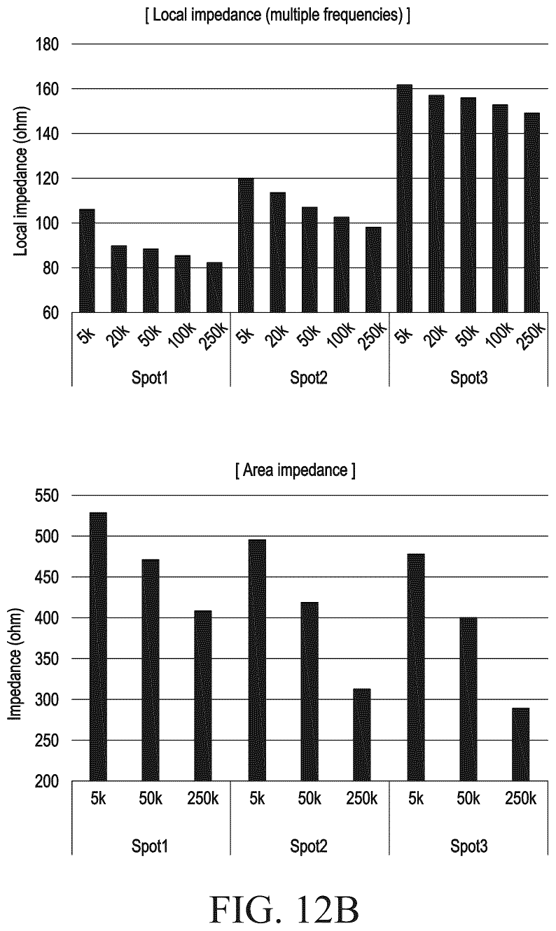

FIGS. 11A, 11B, and 11C are example views illustrating a function or operation of determining the degree of a user's muscle development status based on measured local impedance and area impedance by a wearable device (e.g., the electronic device 101) according to one or more embodiments of the disclosure. FIGS. 12A and 12B are example views illustrating a function or operation of measuring local impedance and area impedance at a plurality of positions using a plurality of frequencies, respectively, by a wearable device according to one or more embodiments of the disclosure.

Referring to FIGS. 11A, 11B, and 11C, the electronic device 101 according to one or more embodiments of the disclosure may estimate the degree of muscle development at the corresponding position based on the measured local impedance. The electronic device 101 according to one or more embodiments of the disclosure may be configured to measure the local impedance using at least four electrodes (e.g., the first electrode 552, the second electrode 554, the third electrode 556, and/or fourth electrode 558). When measuring the local impedance, the electronic device 101 according to one or more embodiments of the disclosure may be configured to apply current (e.g., several mA) to one first pair of electrodes (e.g., the first electrode 552 and the second electrode 554) among the rear electrodes (e.g., the first electrode 552, the second electrode 554, the third electrode 556 and/or the fourth electrode 558) and, for the other remaining second pair of electrodes (e.g., the third electrode 556 and the fourth electrode 558), measure the difference between voltages applied to the other remaining pair of electrodes according to the application of the current. The wearable device 200 according to one or more embodiments of the disclosure may measure the local impedance based on the intensity of the applied current and the voltage difference. The electronic device 101 according to one or more embodiments of the disclosure may measure local impedance value at a designated position of the left arm and local impedance value at a designated position of the right arm. According to one or more embodiments of the disclosure, as muscle mass increases, the impedance value (e.g., the local impedance value) may tend to be measured as relatively low. As illustrated in FIGS. 11B and 11C, when the impedance value of the left arm is larger than the impedance value of the right arm, the electronic device 101 according to one or more embodiments of the disclosure may estimate that the right arm is more developed (e.g., has more muscle mass) than the left arm. The electronic device 101 according to one or more embodiments of the disclosure may compare the measured magnitude of the local impedance value with an initial measurement value and use the comparison result as reference information for identifying the position where the electronic device 101 is worn or determining whether the electronic device 101 is worn at the correct position. Alternatively, the electronic device 101 according to one or more embodiments of the disclosure may measure the area impedance together and estimate the position where the local impedance is measured based on the measured area impedance.

As illustrated in FIGS. 12A and 12B, the electronic device 101 according to one or more embodiments of the disclosure may measure impedance values using a plurality of frequencies, respectively. For example, the electronic device 101 according to one or more embodiments of the disclosure may measure impedance values using a frequency of 5 kHz, a frequency of 20 kHz, a frequency of 50 kHz, a frequency of 100 kHz, and/or a frequency of 250 kHz. The electronic device 101 according to one or more embodiments of the disclosure may control a current source so that, e.g., AC currents including different frequency bands are sequentially applied to at least one electrode (e.g., at least some of the rear electrodes). In measuring the local impedance value and/or the area impedance value, the electronic device 101 according to one or more embodiments of the disclosure may measure the local impedance value and/or the area impedance value based on the reactance value of the inductor, and the reactance value of the capacitor, included in the module configured to measure the impedance including the frequency component. Thus, according to one or more embodiments of the disclosure, when the frequency band for measuring the impedance is changed, the local impedance value and/or the area impedance value may also be changed, and different impedance values may be measured for each frequency band. The electronic device 101 according to one or more embodiments of the disclosure may provide more accurate body information-related feedback to the user by classifying and storing the measured impedance values for each frequency band and identifying the user's body information (e.g., body development degree, body composition) using the stored impedance values for each frequency band.

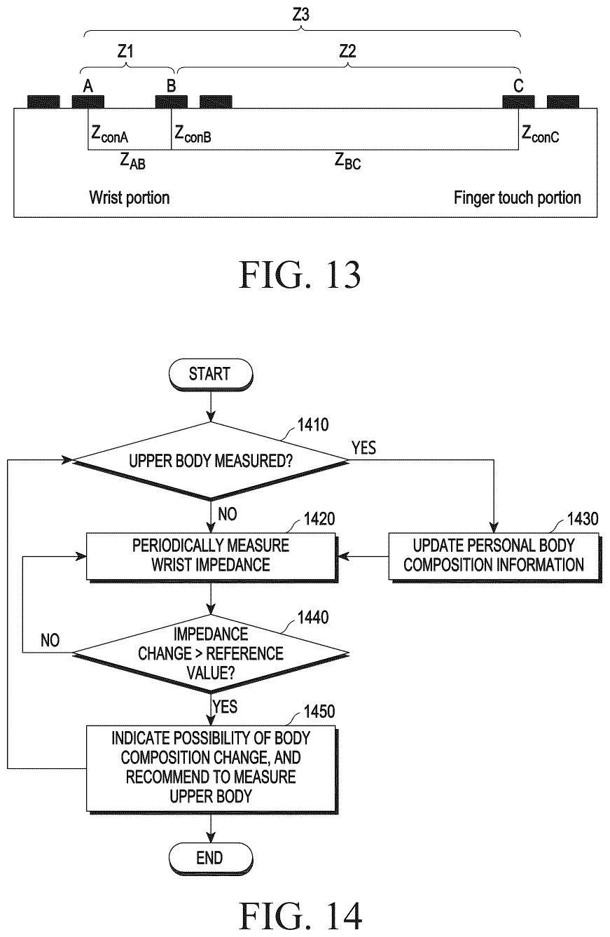

FIG. 13 is a concept view illustrating an electrode structure of a wearable device 200 according to one or more embodiments of the disclosure.

Referring to FIG. 13, the electronic device 101 according to one or more embodiments of the disclosure may measure impedance Z1, impedance Z2, and impedance Z3 through contact of the user's body portion. The impedance Z1 according to one or more embodiments of the disclosure may include the impedance value between the rear electrodes (e.g., the first electrode 552 and the second electrode 554). The impedance Z2 according to one or more embodiments of the disclosure may include the impedance value measured between the second pair (e.g., the third electrode 556 and the fourth electrode 558) of the rear electrodes and the side electrodes. In other words, the impedance value measured between an electrode pair disposed relatively close to the side electrodes may be included. The impedance Z3 according to one or more embodiments of the disclosure may include the impedance value measured between the first pair (e.g., the first electrode 552 and the second electrode 554) of the rear electrodes and the side electrodes. In other words, the impedance value measured between the side electrodes and the electrode pair disposed at a relatively far position may be included. In FIG. 13, the electrode C is illustrated as an electrode corresponding to any one of the side electrodes, but this is for convenience of description, and according to one or more embodiments of the disclosure, the electrode C may include at least one of the side electrodes or all of the side electrodes.

An electronic device 101 according to one or more embodiments of the disclosure may calculate/estimate an impedance ZAB or ZBC for a deeper area than the skin surface based on the measured impedance Z1, impedance Z2, and impedance Z3. The electronic device 101 according to one or more embodiments of the disclosure may calculate contact impedance elements ZconA, ZconB, and ZconC on the electrode/skin surface at each electrode point using the measured impedance Z1, impedance Z2, and impedance Z3 values. For example, the electronic device 101 according to one or more embodiments of the disclosure may calculate/estimate the contact impedance elements ZconA, ZconB, and ZconC at each electrode point using Equation 1 (Eq. 1) and Equation 2 (Eq. 2) below. Here, ZconA and ZconB may be defined as the same value. According to the conventional art, accurate measurements of contact impedance elements (e.g., ZconA, ZconB and/or ZconC) could not be performed, and as a result, accurate measurements of impedance values (e.g., internal impedance values) between electrodes at positions deeper than the skin surface could not be performed. However, since the internal impedance value may be accurately calculated using the electronic device 101 including the electrode structure according to one or more embodiments of the disclosure, it is possible to provide accurate biometric information to the user. Further, the electronic device 101 according to one or more embodiments of the disclosure may be configured to prompt the measurement of the area impedance when the contact impedance element (e.g., ZconA, ZconB, and/or ZconC) is larger than or equal to a designated threshold, or to determine that the measured local impedance value is valid only when the area impedance measurement has been performed. The electronic device 101 according to one or more embodiments of the disclosure may generate a correction table of personalized contact impedance elements (e.g., ZconA, ZconB and/or ZconC) based on historical information about past contact impedance elements (e.g., ZconA, ZconB and/or ZconC).

$${Z}_{conB} = [ ( Z 1 + Z 2 ) - Z 3 ] / 2 \qquad Eq . 1$$

$$Z 1 = {Z}_{conA} + {Z}_{AB} + {Z}_{conB} \qquad Eq . 2$$

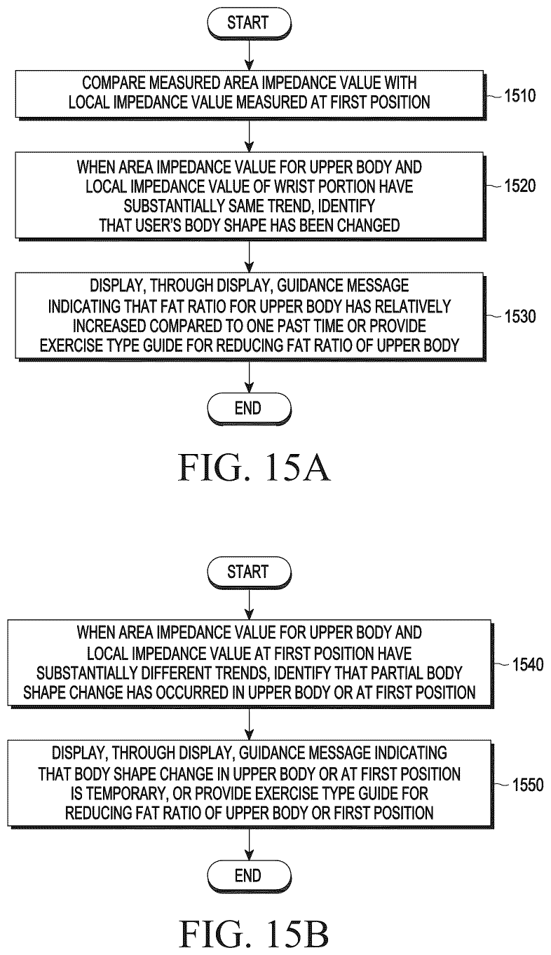

FIG. 14 is an example view illustrating a function or operation of providing information about changes in body composition based on local impedance by a wearable device (e.g., electronic device 101) according to one or more embodiments of the disclosure. FIGS. 15A and 15B are example views illustrating a function or operation of identifying a change in body shape based on measured local impedance and area impedance and providing a guidance message based on the identified change in body shape by a wearable device according to one or more embodiments of the disclosure.

Referring to FIG. 14, the electronic device 101 according to one or more embodiments of the disclosure may determine whether the area impedance for the upper body has been measured in operation 1410. In operation 1420, the electronic device 101 according to one or more embodiments of the disclosure may periodically measure the local impedance for the first position (e.g., wrist) when the area impedance for the upper body is not measured (e.g., when history information for the area impedance for the upper body is not stored in the electronic device 101). The electronic device 101 according to one or more embodiments of the disclosure may determine whether the amount of change in the local impedance exceeds a reference value by comparing the local impedance (e.g., the initial local impedance) at a specific time and the currently measured local impedance in operation 1440. In operation 1450, when determining that the amount of change in the local impedance exceeds the reference value, the electronic device 101 according to one or more embodiments of the disclosure may provide a guide for prompting to measure the area impedance of the upper body. For example, the electronic device 101 according to one or more embodiments of the disclosure may display, through the display, a guidance message to prompt the measurement of the area impedance or a guidance message to indicate that the user is required to contact the side electrode portion to measure the area impedance. In general, a proportional relationship may be established between the body fat percentage and the local impedance, so the electronic device 101 according to one or more embodiments of the disclosure may provide a guide for measuring the area impedance for the upper body. When it is determined that the area impedance for the upper body is measured, the electronic device 101 according to one or more embodiments of the disclosure may update personal body composition information based on the measurement of the area impedance for the upper body in operation 1430. For example, the electronic device 101 according to one or more embodiments of the disclosure may update various body indicators indicating that a change in body shape occurs.

Referring to FIG. 15A, the electronic device 101 according to one or more embodiments of the disclosure may compare the measured area impedance value with the local impedance value measured at the first position (e.g., near the wrist) in operation 1510. For example, the electronic device 101 according to one or more embodiments of the disclosure may compare the trends of the measured area impedance value and the local impedance value measured at the first position (e.g., near the wrist). In operation 1520, based on the comparison result of the trends, the electronic device 101 according to one or more embodiments of the disclosure may identify that the user's body shape has changed when the area impedance value for the upper body and the local impedance value of the wrist have substantially the same trend (e.g., when the difference between the trends is in the designated error range). For example, the electronic device 101 according to one or more embodiments of the disclosure may determine that the body composition (e.g., fat) has increased throughout the upper body when identifying that the area impedance value for the upper body and the local impedance value of the wrist have increased at substantially the same rate. Accordingly, in operation 1530, the electronic device 101 according to one or more embodiments of the disclosure may display, through the display, a guidance message indicating that the fat ratio for the upper body has increased relatively compared to the past time. Alternatively, the electronic device 101 according to one or more embodiments of the disclosure may provide an exercise type guide for reducing the fat ratio for the upper body in operation 1530. Alternatively, in this case, the electronic device 101 according to one or more embodiments of the disclosure may adjust a baseline (e.g., a threshold impedance value) that is a reference for determining a lack or excess of body composition (e.g., body moisture) by recognizing it as an overall body shape change.

Referring to FIG. 15B, in operation 1540, the electronic device 101 according to one or more embodiments of the disclosure may identify that a partial body shape change has occurred in the wrist portion based on the trend comparison results in operation 1510, when the area impedance value for the upper body and the local impedance value of the wrist portion have substantially different trends (e.g., when the area impedance trend difference during a past designated first period and the area impedance trend difference during a second period exhibit substantially the same trend within a designated error range, but the local impedance trend difference exceeds a designated error range so that they exhibit substantially different trends). In this case, in operation 1550, the electronic device 101 according to one or more embodiments of the disclosure may provide a guidance message indicating that a change in the body shape of the wrist occurs at least temporarily. In operation 1540, the electronic device 101 according to one or more embodiments of the disclosure may identify that a body shape change has occurred in the upper body (e.g., the abdomen) when the area impedance value for the upper body and the local impedance value of the wrist portion have substantially different trends (e.g., when the local impedance trend difference during a past designated first period and the local impedance trend difference during a second period exhibit substantially the same trend within a designated error range, but the local impedance trend difference exceeds a designated error range so that they exhibit substantially different trends). In this case, in operation 1550, the electronic device 101 according to one or more embodiments of the disclosure may provide a guidance message indicating that a change in the abdominal body shape occurs. Alternatively, in operation 1550, the electronic device 101 according to one or more embodiments of the disclosure may provide a guidance message as feedback, indicating an exercise type that may reduce the fat ratio of the abdomen. Alternatively, in this case, the electronic device 101 according to one or more embodiments of the disclosure may maintain the baseline (e.g., the threshold impedance value) that is the reference for determining the lack or excess of body composition (e.g., body moisture) by recognizing it as a partial body shape change.

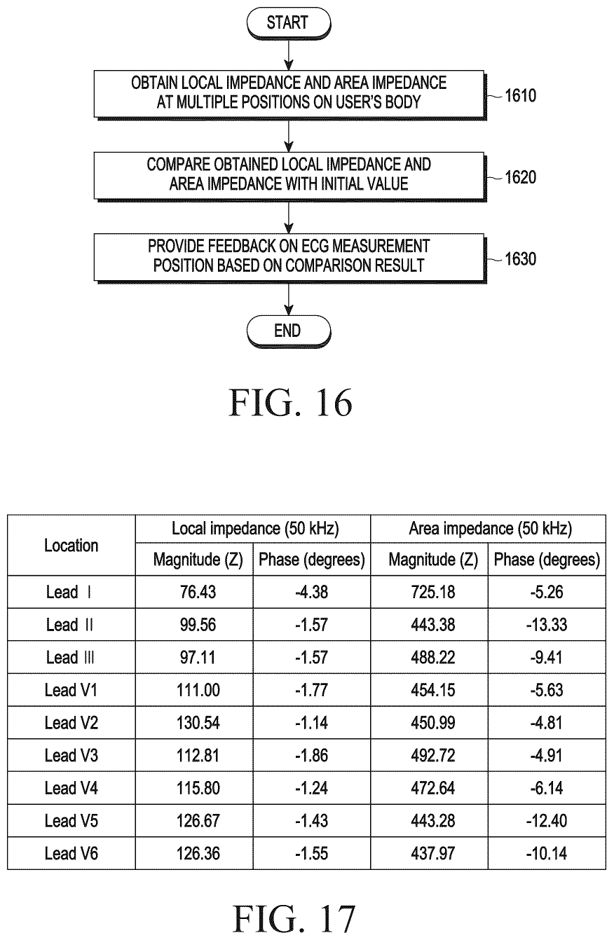

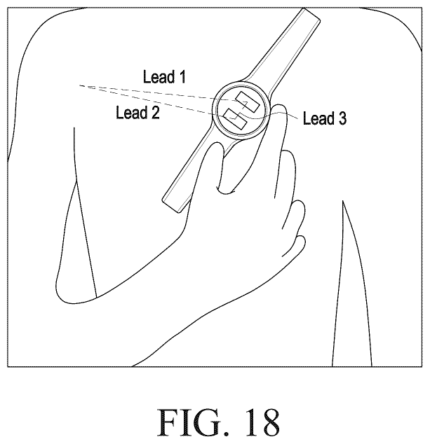

FIG. 16 is an example view illustrating a function or operation of providing feedback on an ECG measurement position by a wearable device (e.g., the electronic device 101) according to one or more embodiments of the disclosure. FIG. 17 is an example view illustrating a table of local impedance and area impedance that a wearable device according to one or more embodiments of the disclosure references to provide feedback on an ECG measurement position. FIG. 18 is an example view illustrating a scheme for simultaneously measuring leads 1, 2, and 3 for ECG measurement using a wearable device according to one or more embodiments of the disclosure.

Referring to FIGS. 16, 17, and 18, the electronic device 101 according to one or more embodiments of the disclosure may obtain the local impedance and the area impedance at a plurality of positions of the user's body in operation 1610. The electronic device 101 according to one or more embodiments of the disclosure may measure the local impedance and the area impedance in a position or state for measuring lead 1 (e.g., a state in which the electronic device 101 is worn on the wrist, and the user's right finger is in contact with at least one of the side electrodes), a position or state for measuring lead 2 (e.g., a state in which at least some of the rear electrodes of the electronic device 101 are in contact with the lower abdomen, and the user's right finger is in contact with at least one of the side electrodes), a position or state for measuring lead 3 (e.g., a state in which at least some of the rear electrodes of the electronic device 101 are in contact with the lower abdomen, and the user's left finger is in contact with at least one of the side electrodes), a position or state for measuring lead V1 (e.g., a state in which at least some of the rear electrodes of the electronic device 101 are in contact with the right chest, and the user's right finger is in contact with at least one of the side electrodes), a position or state for measuring lead V2 (e.g., a state in which at least some of the rear electrodes of the electronic device 101 are in contact with the left chest, and the user's right finger is in contact with at least one of the side electrodes) and/or in a position or state for measuring leads V3, V4, V5, and V6. In this case, in order to measure the local impedance and the area impedance, the user's finger may be required to contact at least two side electrodes. Information about the plurality of positions according to one or more embodiments of the disclosure may be input by the user or may be previously stored in the electronic device 101. When the information about the plurality of positions according to one or more embodiments of the disclosure is previously stored in the electronic device 101, the previously stored information about the positions may be provided (e.g., displayed through the display). In order to determine whether the electronic device 101 is correctly placed at each of the plurality of positions, the electronic device 101 may obtain an acknowledgment indicating that the electronic device 101 is correctly placed at each of the plurality of positions from the user through the electronic device 101, and when the acknowledgment is obtained, the electronic device 101 may determine that the electronic device 101 is placed at the correct position. Alternatively, the electronic device 101 according to one or more embodiments of the disclosure may determine whether the electronic device 101 is placed at the correct position using an impedance value (e.g., the local impedance value and/or the area impedance value) corresponding to information about the standard body shape determined based on the user's body information. For example, the electronic device 101 according to one or more embodiments of the disclosure may determine whether the electronic device 101 is placed at the correct position by comparing the area impedance value (e.g., the reference local impedance value and/or the reference area impedance value) stored in the electronic device 101 with the area impedance value newly measured in the lower abdomen.

The electronic device 101 according to one or more embodiments of the disclosure may compare the obtained local impedance and area impedance with an initial value in operation 1620. For example, the electronic device 101 according to one or more embodiments of the disclosure may compare the local impedance values and area impedance values first measured in the positions or states for measuring lead 1 to lead 3 or lead V1 to lead V6, with the obtained local impedance and area impedance values. For example, the electronic device 101 according to one or more embodiments of the disclosure may compare the respective impedance values by determining whether the respective impedance values are included in the designated error range. As illustrated in FIG. 17, the electronic device 101 according to one or more embodiments of the disclosure may determine whether the electronic device 101 is at the right position for measuring lead 1 to lead V6 by comparing the magnitude and phase of the local impedance and the magnitude and phase of the area impedance at a designated position (e.g., the position for measuring lead 1) with the reference local impedance value and/or the reference area impedance value. When each impedance value is included in the designated error range, the electronic device 101 may determine that the electronic device 101 is placed at the correct position for measuring lead 1 to lead 3, or the lead V1 to lead V6. As such, by determining the measurement position based on up to four types of characteristics, the electronic device 101 may also accurately determine whether the electronic device 101 is placed at the positions corresponding to leads 2 and 3 configured to measure by merely switching the left/right hands at substantially the same measurement position.

The electronic device 101 according to one or more embodiments of the disclosure may provide feedback on the ECG measurement position based on the comparison result, in operation 1630. The first position (e.g., the position where the ECG was initially measured) according to one or more embodiments of the disclosure may be input through the electronic device 101 by the user, pre-stored in the electronic device 101, and/or estimated based on an impedance value corresponding to the standard body shape by the electronic device 101. When it is determined that the electronic device 101 is positioned at the correct position, the electronic device 101 according to one or more embodiments of the disclosure may perform at least one operation for measuring the ECG at the corresponding position. However, the electronic device 101 according to one or more embodiments of the disclosure may provide a notification message to change the position of the electronic device 101 when determining that the electronic device 101 is not placed at the correct position.

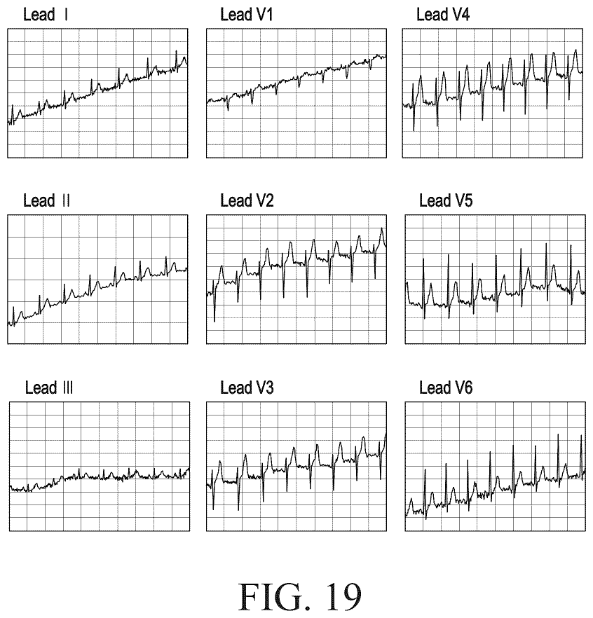

FIG. 19 is an example view illustrating ECG signals exhibiting different waveforms obtained at different positions on a user's body, according to one or more embodiments of the disclosure.

Referring to FIGS. 18 and 19, it is possible to simultaneously measure the signals corresponding to the lead 1, lead 2, and lead 3 directions that are required for ECG measurement using a plurality of electrodes (e.g., the first electrode 552, the second electrode 554, the third electrode 556, and/or the fourth electrode 558) according to one or more embodiments of the disclosure. For example, the electronic device 101 according to one or more embodiments of the disclosure may obtain an ECG signal corresponding to the lead 1 direction by measuring the impedance between at least one of the side electrodes and the first electrode 552. Further, the electronic device 101 according to one or more embodiments of the disclosure may obtain an ECG signal corresponding to the lead 2 direction by measuring the impedance between at least one electrode (e.g., first electrode 552) and another electrode (e.g., second electrode 554). The electronic device 101 according to one or more embodiments of the disclosure may obtain an ECG signal corresponding to the lead 3 direction by measuring the impedance between at least one of the side electrode portions and the second electrode 554. The electronic device 101 according to one or more embodiments of the disclosure may substantially simultaneously perform the function or operation of obtaining the ECG signal corresponding to the lead 1 direction, the function or operation of obtaining the ECG signal corresponding to the lead 2 direction, and the function or operation of obtaining the ECG signal corresponding to the lead 3 direction. In this case, the electronic device 101 according to one or more embodiments of the disclosure may be positioned near the user's heart (e.g., left chest) to obtain each ECG signal. According to the above-described embodiments related to FIGS. 18 and 19, ECG signals representing different waveforms at different positions may be obtained, as illustrated in FIG. 19.

A wearable device according to one or more embodiments of the disclosure may comprise a housing, a display disposed on the housing to be at least partially exposed, a plurality of first electrodes disposed on a first surface of the housing, a plurality of second electrodes disposed on a second surface of the housing, and at least one processor disposed inside the housing. The at least one processor may be configured to detect a contact of the plurality of first electrodes to a first position on a user's body, detect a contact of a portion of the user's body to the plurality of second electrodes while the plurality of first electrodes contact the first position, measure both a local impedance at the first position and an area impedance including the first position based on the detection of the contact of the plurality of first electrodes and the plurality of second electrodes, and provide information about the user's body through the display based on the local impedance and the area impedance.

In a computer-readable, non-transitory recording medium configured to store a plurality of instructions, according to one or more embodiments of the disclosure, the plurality of instructions include instructions configured to, when executed by at least one processor of a wearable device, enable the wearable device to detect a contact of a plurality of first electrodes of the wearable device to a first position on a user's body, detect a contact of a portion of the user's body portion to a plurality of second electrodes of the wearable device while the plurality of first electrodes contact the first position, measure both a local impedance at the first position and an area impedance including the first position based on the detection of the contact of the plurality of first electrodes and the plurality of second electrodes, and provide information about the user's body through a display of the wearable device based on the local impedance and the area impedance.

A wearable device according to one or more embodiments of the disclosure may comprise a housing, a display disposed on the housing to be at least partially exposed, a plurality of first electrodes disposed on a first surface of the housing, a plurality of second electrodes disposed on a second surface of the housing, and at least one processor disposed inside the housing, wherein the at least one processor is configured to measure, using the plurality of first electrodes and the plurality of second electrodes, a local impedance and an area impedance at a plurality of positions, respectively, on a user's body, based on the local impedance and the area impedance, determine whether at least one position among the plurality of positions is a correct position or not; and when the at least one position among the plurality of positions is the correct position, measure the ECG signal at the plurality of positions where the local impedance and the area impedance have been measured.

The electronic device according to various embodiments may be one of various types of electronic devices. The electronic devices may include, for example, a portable communication device (e.g., a smart phone), a computer device, a portable multimedia device, a portable medical device, a camera, a wearable device, or a home appliance. The electronic devices according to an embodiment are not limited to those described above.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used herein, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The storage medium readable by the machine may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program products may be traded as commodities between sellers and buyers. The computer program product may be distributed in the form of a machine-readable storage medium (e.g., compact disc read only memory (CD-ROM)), or be distributed (e.g., downloaded or uploaded) online via an application store (e.g., Play Store™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to an embodiment of the disclosure, each component (e.g., a module or a program) of the above-described components may include a single entity or multiple entities. Some of the plurality of entities may be separately disposed in different components. According to an embodiment of the disclosure, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to an embodiment of the disclosure, operations performed by the module, the program, or another component may be carried out sequentially, in parallel, repeatedly, or heuristically, or one or more of the operations may be executed in a different order or omitted, or one or more other operations may be added.

What is claimed is:

1. A wearable device comprising:
a housing;
a display disposed on the housing and at least partially exposed from the housing;
a plurality of first electrodes disposed on a first surface of the housing;
a plurality of second electrodes disposed on a second surface of the housing; and
at least one processor disposed inside the housing, wherein the at least one processor is configured to:
detect a first contact of the plurality of first electrodes to a first position on a user's body;
detect a second contact of a portion of the user's body to the plurality of second electrodes while the plurality of first electrodes contact the first position;
based on detecting the first contact and the second contact, measure both a local impedance at the first position and an area impedance of a combination of the first position and one or more other positions on the user's body; and
based on the local impedance and the area impedance, provide, through the display, information about the user's body.

2. The wearable device of claim 1, wherein the plurality of first electrodes include pairs of electrodes.

3. The wearable device of claim 1, wherein the at least one processor is further configured to, based on history information about the local impedance measured at the first position, control the display to provide a guide of a measurement position.

4. The wearable device of claim 1, wherein the at least one processor is further configured to, based on the local impedance, perform a scoring operation on the first position.

5. The wearable device of claim 1, wherein the at least one processor is further configured to control the display to provide a guide of a measurement of the area impedance based on determining that an amount of change in the local impedance of the first position exceeds a designated threshold amount of change.

6. The wearable device of claim 1, wherein the at least one processor is further configured to:
determine a body condition at the first position and a second position of the user's body different from the first position and based on the local impedance, the one or more other positions of the user's body comprise the second position; and
provide, through the display, an indication of the body condition.

7. The wearable device of claim 1, wherein the at least one processor is further configured to measure the local impedance at the first position using a plurality of frequencies of alternating current (AC) applied to one or more of the plurality of first electrodes.

8. The wearable device of claim 1, wherein the at least one processor is further configured to:
determine whether the local impedance measured at the first position exceeds a designated threshold impedance; and
based on determining that the local impedance exceeds the designated threshold impedance, output a notification message indicating that a condition of the user's body is changed.

9. The wearable device of claim 1, wherein the at least one processor is further configured to, based on a result of the local impedance and the area impedance, change a baseline of a body composition trend from a first baseline to a second baseline.

10. A wearable device comprising:
a housing;
a display disposed on the housing and at least partially exposed;
a plurality of first electrodes disposed on a first surface of the housing;
a plurality of second electrodes disposed on a second surface of the housing; and
at least one processor disposed inside the housing, wherein the at least one processor is configured to:
measure, using the plurality of first electrodes and the plurality of second electrodes, a local impedance and an area impedance at a plurality of positions, respectively, on a user's body, the local impedance being of a first position on the user's body, the area impedance being of a combination of the first position and one or more other positions on the user's body, and the plurality of positions comprises the first position and the one or more other positions;
based on the local impedance and the area impedance, determine whether at least one position among the plurality of positions is a predetermined position; and
based on determining that the at least one position among the plurality of positions is the predetermined position, measure an electrocardiogram (ECG) signal at the plurality of positions.

11. The wearable device of claim 10, wherein the at least one processor is further configured to, based on determining that the first position does not correspond to the predetermined position, output a message guiding the wearable device to be moved to another position of the user's body.

12. A non-transitory computer-readable recording medium storing a plurality of instructions, wherein the plurality of instructions, when executed by at least one processor of a wearable device, cause the wearable device to:
detect a first contact of a plurality of first electrodes of the wearable device to a first position on a user's body;
detect a second contact of a portion of the user's body to a plurality of second electrodes of the wearable device while the plurality of first electrodes contact the first position;
based on detecting the first contact and the second contact, measure both a local impedance at the first position and an area impedance of a combination of the first position and one or more other positions of the user's body; and
based on the local impedance and the area impedance, provide, through a display of the wearable device, information about the user's body.

13. The non-transitory computer-readable recording medium of claim 12, wherein the plurality of first electrodes include pairs of electrodes.

14. The non-transitory computer-readable recording medium of claim 12, wherein the instructions further cause the wearable device to, based on history information about the local impedance measured at the first position, control the display of the wearable device to provide a guide of a measurement position.

15. The non-transitory computer-readable recording medium of claim 12, wherein the instructions further cause the wearable device to, based on the local impedance, perform a scoring operation on the first position.

16. The non-transitory computer-readable recording medium of claim 12, wherein the instructions further cause the wearable device to control the display of the wearable device to provide a guide of a measurement of the area impedance based on determining that an amount of change in the local impedance of the first position exceeds a designated threshold amount of change.

17. The non-transitory computer-readable recording medium of claim 12, wherein the instructions further cause the wearable device to:
determine a body condition at the first position and a second position of the user's body different from the first position and based on the measured local impedance, the one or more other position of the user's body comprise the second position; and
provide, through the display, an indication of the body condition.

18. The non-transitory computer-readable recording medium of claim 12, wherein the instructions further cause the wearable device to measure the local impedance at the first position using a plurality of frequencies of alternating current (AC) applied to one or more the plurality of first electrodes.

19. The non-transitory computer-readable recording medium of claim 12, wherein the instructions further cause the wearable device to:
determine whether the local impedance measured at the first position exceeds a designated threshold impedance; and
based on determining that the local impedance exceeds the designated threshold impedance, output a notification message indicating that a conditions of the user's body is changed.

20. The non-transitory computer-readable recording medium of claim 12, wherein the instructions further cause the wearable device to, based on a result of the local impedance and the area impedance, change a baseline of a body composition trend from a first baseline to a second baseline.
